(19) United States
(12) Patent Application Publication
(10) Pub. No.: US 2005/0020615 A1
(43) Pub. Date: Jan. 27, 2005

(54) CCI-779 lyophilized formulations

(75) Inventor: Joseph T. Rubino, Towaco, NJ (US)

Correspondence Address: HOWSON AND HOWSON;CATHY A. KODROFF, ONE SPRING HOUSE CORPORATE CENTER, BOX 457, SPRING HOUSE, PA, 19477, US

(73) Assignee: Wyeth, Madison, NJ (US)

(21) Appl. No.: 10/894,210

(22) Filed: Jul. 19, 2004

Related U.S. Application Data
(60) Provisional application No. 60/490,293, filed on Jul. 25, 2003.

(51) Int. Cl. A61K 31/4745

(52) U.S. Cl. 514/291

(57) ABSTRACT

Lyophilized CCI-779 formulations composed of CCI-779 and t-butyl alcohol or ethanol are described. Also provided are solutions useful in preparing said lyophilized CCI-779 formulations, methods of reconstituting same, and uses for same.

### CROSS-REFERENCE TO RELATED APPLICATION

This application claims the benefit of U.S. Patent Application No. 60/490,293, filed Jul. 25, 2003.

### BACKGROUND OF THE INVENTION

CCI-779 is the 42-bis-hydroxymethylpropionic acid ester of rapamycin that is being evaluated in clinical trials for activity against cancer, multiple sclerosis and rheumatoid arthritis. CCI-779 exhibits cytostatic, as opposed to cytotoxic properties, and may delay the time to progression of tumors or time to tumor recurrence. CCI-779 is considered to have a mechanism of action that is similar to that of sirolimus (rapamycin). CCI-779 binds to and forms a complex with the cytoplasmic protein FKBP, which inhibits an enzyme, mTOR (mammalian target of rapamycin, also known as FKBP12-rapamycin associated protein [FRAP]). Inhibition of mTOR's kinase activity inhibits a variety of signal transduction pathways, including cytokine-stimulated cell proliferation, translation of mRNAs for several key proteins that regulate the G1 phase of the cell cycle, and IL-2-induced transcription, leading to inhibition of progression of the cell cycle from Gi to S. The mechanism of action of CCI-779 that results in the G1 to S phase block is novel for an anticancer drug.

In vitro, CCI-779 has been shown to inhibit the growth of a number of histologically diverse tumor cells. Central nervous system (CNS) cancer, leukemia (T-cell), breast cancer, prostate cancer, and melanoma lines were among the most sensitive to CCI-779. The compound arrested cells in the G1 phase of the cell cycle.

In vivo studies in nude mice have demonstrated that CCI-779 has activity against human tumor xenografts of diverse histological types. Gliomas were particularly sensitive to CCI-779 and the compound was active in an orthotopic glioma model in nude mice. Growth factor (platelet-derived)-induced stimulation of a human glioblastoma cell line in vitro was markedly suppressed by CCI-779. The growth of several human pancreatic tumors in nude mice as well as one of two breast cancer lines studied in vivo also was inhibited by CCI-779.

The physical-chemical properties of CCI-779 that result in challenges to the successful formulation of oral and liquid dosage forms include poor solubility in water and chemical instability due to several mechanisms.

### SUMMARY OF THE INVENTION

The present invention provides lyophilized CCI-779 formulations that overcome the undesirable physical chemical properties of prior formulations of CCI-779. The resulting material can be used to produce dosage forms that are suitable for administration via parenteral routes or as an intermediate to be delivered orally.

Other aspects and advantages of the present invention will be readily apparent from the following detailed description.

### DESCRIPTION OF THE INVENTION

The present invention provides pre-lyophilization formulations that provide freeze-dried CCI-779 of the invention with improved potency retention and stability under storage conditions. More particularly, using the pre-lyophilization formulations of the invention, freeze-dried CCI-779 has been found to retain greater than 95% initial potency after one month storage at 40° C. and after six months storage at room temperature. The present invention also provides reconstituted CCI-779 formulations suitable for delivery parenterally or by other routes of delivery.

The preparation of CCI-779 is described in U.S. Pat. No. 5,362,718, which is hereby incorporated by reference. A regioselective preparation of CCI-779 is described in U.S. Pat. No. 6,277,983, which is hereby incorporated by reference.

A pre-lyophilization solution of CCI-779 of the invention is formed by dissolving CCI-779 in a suitable organic solvent or mixture of organic solvent and water. Suitably, the solvent is sufficiently volatile to be removed under typical temperature and pressure conditions that are used in a commercial freeze dryer. In addition, the solubility of CCI-779 in the organic solvent or solvent-water mixture is sufficiently high to produce material that is concentrated enough to permit practical applications of the drug. Typically, the concentration of CCI-779 in the pre-lyophilized solutions range from 0.1 to 250 mg/mL to provide a lyophilized form of CCI-779 which is suitable for preparing doses of CCI-779 of from 1 to 500 mg. Examples of effective solvents include dimethysulfoxide, acetonitrile, ethanol, isopropanol, t-butyl alcohol, and blends containing same alone or with water. Of these solvents, t-butyl alcohol is preferred. Ethanol is also expected to be particularly desirable because, like t-butyl alcohol, it has a low relative order of toxicity and can be combined with water and removed under vacuum at low temperatures.

These solvents, or blends containing these solvents are present in an amount of about 30% to about 40%, to about 50%, to about 60%, to about 70%, to about 80%, to about 90%, to about 95%, to about 100% v/v although lower amounts of the individual solvents may be selected to provide a blend to provide a total solvent amount in the provided range. Water may be present in an amount of about 0% to about 70% v/v of a solvent blend. Preferably, however, the solvent blend contains less than 40% v/v (i.e., 0% w/v to 40% v/v water), and preferably, less than 30% v/v water (i.e., 0% v/v to 30% v/v water) based on the v/v % of the total solution.

Desirably, when a significant amount of water (e.g,. 40% v/v or greater) is present in the solvent blend of the pre-lyophilized solution, it is advantageous to adjust the pH to 4 to 6 as this is the range of maximum stability for CCI-779. In one embodiment, the pH is adjusted to a pH of about 5.5.

In certain embodiments, the pre-lyophilization solution may further contain bulking agents or antioxidants. These components can be readily selected by one of skill in the art in view of the selected solvent or solvent blend. In particular, the solubility of typical water-soluble bulking agents such as sugars or polyols is reduced by the presence of organic solvents. In these embodiments, a blend of organic solvent and water are used and the composition adjusted in order to balance an adequate concentration of drug with an effective concentration of added substance. Suitable bulking agents include mannitol and sucrose. Additional, optional, materials include polyvinylpyrrolidone, dextran, starch, lactose, trehalose or hydroxyethylstarch and glycerol. Combinations of the above bulking agents can be used. Bulking agents can be used in a range of 0.5 to 10% w/v in the pre-lyophilized solution.

Optionally, the pre-lyophilization solutions of the invention contain an antioxidant component(s) in a concentration ranging from 0.001% to 1% w/v, or 0.01% to 0.5% w/v, although lower or higher concentrations may be desired. Examples of suitable antioxidants and optimal concentrations include BHT (0.005-0.02% w/v), BHA (0.005-0.02% w/v), alpha-tocopherol (0.05-0.075% w/v), ascorbic acid (0.02-0.5% w/v), erythorbic acid (0.1-1.0% w/v), dithiothreitol (0.01-0.1% w/v), dithioerythreitol (0.01-0.1% w/v), glutathione (0.01-0.1% w/v), ascorbyl palmitate ((0.01-0.02% w/v), monothioglycerol (0.1-0.5% w/v), propylgallate (0.05-0.1% w/v), sodium bisulfite (0.05-1.0% w/v), sodium metabisulfite (0.025-0.1% w/v).

In certain embodiments, the antioxidant component of the formulation of the invention also exhibits chelating activity. Examples of such chelating agents include, e.g., citric acid, succinic acid, malic acid, maleic acid, malonic acid, glutaric acid, adipic acid. Other acidifying agents which inhibit metal-catalyzed reactions but do not necessarily act as chelating agents include acetic acid, and ascorbic acid (0.001-0.0.1% w/v) (which may function as both a classic antioxidant and inhibit metal catalysis in the present formulations). Other chelating agents include such materials as are capable of binding metal ions in solution, such as ethylene diaminetetraacetic acid (EDTA) and its salts (0.002-0.1% w/v), glycine, glutamic acid or other amino acids (0.002-0.1% w/v) are capable of enhancing the stability of CCI-779.

In some embodiments, components with chelating activity are included in the formulations of the invention as the sole “antioxidant component”. Typically, such metal-binding components, when acting as chelating agents are used in the lower end of the range of concentrations for the antioxidant component provided herein. In one example, citric acid enhanced the stability of CCI-779 when used at a concentration of less than 0.01% w/v. Higher concentrations result in less stable solutions and thus, less desirable for products to be subject to long-term storage in liquid form. Additionally, such chelating agents may be used in combination with other antioxidants as part of the antioxidant component of the invention. For example, an acceptable formulation may contain both citric acid and d,l-α-tocopherol. Optimal concentrations for the selected antioxidant(s) can be readily determined by one of skill in the art, based upon the information provided herein. All percentages are expressed a % w/v in the pre-lyophilized solution.

Desirably, the pre-lyophilization solution has a pH in the range of 4 to 6, which has been found by the inventor to improve the stability of CCI-779. Depending upon the components of the pre-lyophilization solution, the pH can be adjusted using any suitable inorganic or organic acid, or base, as needed. Thereafter, the pre-lyophilization solution is subject to freeze-drying.

Freeze-drying can be performed using commercial freeze-dryers, such as are available from a variety of sources using manufacturer recommended settings. Desirably, the product is freeze dried so that the lyophilized product contains less than 1% w/v solvent/diluent. In one example, the product is loaded at about 20° C. frozen at about −40° C. at approximately 30 degrees/hour; held at −40° C. for six hours, and the frozen solution is thermally treated by raising the shelf temperature to −20° C. and holding for 2 to 8 hours. Alternatively, the frozen solution can be thermally treated by cycling the temperature from −40° C. to −5° C. and back to −20° C. Thereafter, the condenser can be started and the vacuum adjusted (e.g., to 100 mTorr) and the shelf temperature is raised to +10° C. Optionally, when the product temperature reaches +10° C., the product is subjected to secondary drying. Such secondary drying can begin when the shelf temperature has reached about 40° C. Secondary drying is performed under pressure, e.g., about 100 mTorr, overnight (e.g., about 12 to 18 hours), or for up to about 24 hours. Alternatively, this step may be performed for a shorter or longer time. Suitably, the freeze-drying results in a product having residual solvent in an amount of less than 1% by weight of the final weight of solids in the lyophilized CCI-779. In addition or alternatively to the second step, other processing techniques can be used to further reduce the residual solvent in the resulting lyophilized material. Such processing techniques include nitrogen sweeps.

Advantageously, the lyophilized CCI-779 of the invention retains greater than 95% potency for an extended period of time under a variety of storage conditions. This lyophilized composition is useful for preparing a variety of dosage forms for delivery to subject, and is particularly advantageous for formulation of liquid and oral dosage forms.

When preparing freeze-dried CCI-779 for reconstitution, a suitable solvent is selected. An effective solvent for reconstitution is biocompatible, dissolves adequate quantities of drug in relatively small volumes and prevents precipitation of the drug during injection into body fluids or dilution in intravenous infusion solutions. In one embodiment, parenterally acceptable amphiphilic compounds are combined with water, organic solvents or a mixture of water with organic solvents. Examples of suitable amphiphilic compounds includes polysorbate 20, 60 or 80, ethoxylated oils, such as PEG-35 castor oil (e.g. Cremophor EL), fatty acid-PEG esters, such as Solutol HS, ], vitamin E tocopherol propylene glycol succinate (Vitamin E TPGS), sucrose-fatty acid esters, bile salts, phospholipids and combinations of bile salts with phospholipids. The concentration of amphiphile can range from 2% to 100% w/v in the reconstitution solvent. Alternatively, in certain embodiments, the amphiphile can be incorporated with CCI-779 in the pre-lyophilization formulation. In such embodiments, reconstitution can be accomplished using either water or a combination of water and organic solvent.

When CCI-779 is reconstituted according to this invention, the reconstituted formulation can contain concentrations of CCI-779 from 0.05 mg/mL, from 2.5 mg/mL, from 5 mg/mL or from 10 mg/mL up to approximately 50 mg/ml. The concentrate car be mixed with the diluent up to approximately 1 part concentrate to 1 part diluent, to give formulations having concentrations of CCI-779 from 1 mg/mL, from 5 mg/mL, from 10 mg/mL, from 20 mg/mL, up to approximately 25 mg/ml. This invention also covers formulations having lesser concentrations of CCI-779 in the cosolvent concentrate, and formulations in which one part of the concentrate is mixed with greater than 1 part of the diluent, e.g., concentrate: diluent in a ratio of about 1:1.5, 1:2, 1:3, 1:4 or 1:5 v/v, and so on, to CCI-779 formulations having a CCI-779 concentration down to the lowest levels of detection. A suitable diluent can readily be selected by one of skill in the art, in view of the route of delivery. For example, the diluent can be aqueous, primarily aqueous, e.g., glucose solution, saline, buffered saline, 0.9% sodium chloride injection, 5% dextrose injection, lactated ringers injection, or non-aqueous.

In one embodiment, lyophilized CCI-779 is reconstituted for administration by the parenteral route with a diluent containing 5 to 10% w/v polysorbate 80, or about 8% w/v polysorbate 80, 35 to 45% w/v dehydrated alcohol or about 40% w/v dehydrated alcohol, and the remainder water to produce a concentrate having 5 to 10 mg/mL CCI-779. Alternatively, lyophilized CCI-779 is reconstituted for administration by the parenteral route using about 5 to 10% w/v polysorbate 80 and water. Optionally, the reconstituted concentrate is diluted with a sodium chloride solution to provide the desired concentration of CCI-779 for injection.

The reconstituted formulations of this invention can be used to produce a parenteral dosage form. Such a dosage form may be suitable for administration by either direct injection or by addition to sterile infusion fluids for intravenous infusion.

Examples of suitable parenteral dosage forms are provided in U.S. patent application Ser. No. 10/626,943 and its corresponding International Patent Application No. WO 2004/011000.

Particularly suitable injectable formulations for rapamycin 42-ester with 3-hydroxy-2-(hydroxymethyl)-2-methylpropionic acid are disclosed in U.S. patent application Ser. No. 10/626,943 and International Patent Publication WO 2004/011000, which are hereby incorporated by reference.

In this embodiment, the injectable formulation useful in the invention provides a CCI-779 cosolvent concentrate containing an parenterally acceptable solvent and an antioxidant as described above and a parenteral formulation containing CCI-779, composed of CCI-779, an parenterally acceptable cosolvent, an antioxidant, a diluent solvent, and a surfactant. Any given formulation useful in this invention may contain multiple ingredients of each class of component. For example, a parenterally acceptable solvent can include a non-alcoholic solvent, an alcoholic solvent, or mixtures thereof. Examples of suitable non-alcoholic solvents include, e.g., dimethylacetamide, dimethylsulfoxide, or mixtures thereof. “An alcoholic solvent,” may contain one or more alcohols as the alcoholic solvent component of the formulation. Examples of solvents useful in the formulations invention include, without limitation, ethanol, propylene glycol, polyethylene glycol 300, polyethylene glycol 400, polyethylene glycol 600, polyethylene glycol 1000, or mixtures thereof. Ethanol and propylene glycol are particularly desirable because degradation via oxidation and lactone cleavage occurs to a lower extent for these cosolvents. Further, ethanol and propylene glycol can be combined.

Advantageously, in certain embodiments of the parenteral formulations useful in the invention, precipitation of CCI-779 upon dilution with aqueous infusion solutions or blood is prevented through the use of a surfactant contained in the diluent solution. One particularly desirable surfactant is polysorbate 20 or polysorbate 80. However, one of skill in the art may readily select other suitable surfactants. Other components of the diluent may include water, ethanol, polyethylene glycol 300, polyethylene glycol 400, polyethylene glycol 600, polyethylene glycol 1000, or blends containing one or more of these polyethylene glycols, propylene glycol and other parenterally acceptable cosolvents or agents to adjust solution osmolarity such as sodium chloride, lactose, mannitol or other parenterally acceptable sugars, polyols and electrolytes. It is expected that the surfactant will comprise 2 to 100% w/v of the diluent solution, 5 to 80% w/v, 10 to 75% w/v, 15 to 60% w/v, and preferably, at least 5% w/v, or at least 10% w/v, of the diluent solution.

A parenteral formulation useful in the invention can be prepared as a single solution, or preferably can be prepared as a cosolvent concentrate containing CCI-779, an alcoholic solvent, and an antioxidant, which is subsequently combined with a diluent that contains a diluent solvent and suitable surfactant.

The parenteral formulations useful in this invention can be used to produce a dosage form that is suitable for administration by either direct injection or by addition to sterile infusion fluids for intravenous infusion.

In some cases it may be desirable to administer the compounds directly to the airways in the form of an aerosol.

Alternatively, the reconstituted formulations of the invention can be used to produce a dosage which is suitable for oral administration. Examples of suitable oral dosage forms are provided in U.S. patent application Ser. No. 10/663,506 and its corresponding International Patent Application WO 2004/026280; U.S. Pat. No. 6,197,781, and U.S. Pat. No. 6,004,973, which are incorporated herein by reference. Such an oral formulation contains contains CCI-779, a water soluble polymer, a pH modifying agent, a surfactant, and an antioxidant.

The compositions of the invention may be produced in the form of a kit of parts. Such a kit is useful for preparing an aqueous pharmaceutical composition. Typically, the kit will contain, at a minimum, a first container having the lyophilized CCI-779 composition of the invention and a second container having a physiologically acceptable solvent therefore. Other components may include vials, stirrers, lids, instructions for reconstititution, mixing, storage and/or, use. Optionally, other active ingredients to be administered in a regimen with the lyophilized or reconstituted CCI-779 may also be provided. The invention also includes a pharmaceutical pack containing a course of treatment for one individual mammal, wherein the pack contains CCI-779 and one or more of the kit components described above.

The following examples are illustrative of the present invention. The present invention is not limited to the percentages, components and techniques described herein.

### EXAMPLES

Examples 1 to 8 provide illustrative pre-lyophilization formulations of the invention which have been freeze dried according to the method of the invention.

### Example 1

| | |
|---|---|
| CCI-779 | 100 mg |
| 70% t-butyl alcohol in water | q.s. 1 mL |

The above solution was filtered, filled into glass vials and freeze dried to remove the t-butyl alcohol-water mixture. The vials were back-filled with nitrogen gas prior to stoppering. X-ray diffraction patterns indicated that the resulting material was largely amorphous. The freeze-dried material was found to retain greater than 98% potency after 5 months storage at 40° C.

### Example 2

| | |
|---|---|
| CCI-779 | 25 mg |
| Mannitol | 2% w/v |
| 0.01 N HCl adjust pH to 5.5 | q.s. |
| 60% t-butyl alcohol in water | q.s. 1 mL |

The above solution was filtered, filled into glass vials and freeze dried to remove the t-butyl alcohol-water mixture. The vials were back-filled with nitrogen gas prior to stoppering. X-ray diffraction patterns closely matched freeze dried mannitol placebo formulations with no evidence of crystalline drug. The freeze-dried material was found to retain greater than 95% initial potency after one month storage at 40° C. and after 6 months storage at room temperature.

### Example 3

| | |
|---|---|
| CCI-779 | 10 mg |
| Mannitol | 2-5% w/v |
| 0.01 N HCl adjust pH to 5.5 | q.s |
| 40% v/v t-butyl alcohol in | q.s. 1 mL |
| water | |

In the above formula, the lower concentration of t-butyl alcohol permits a wider range of the bulking agent, mannitol, to be incorporated into the pre-lyophilized solution. In one experiment, a solution containing 2% w/v mannitol was filtered, filled into glass vials and freeze dried to remove the t-butyl alcohol-water mixture. The vials were back-filled with nitrogen gas prior to stoppering. X-ray diffraction patterns closely matched freeze dried mannitol placebo formulations with no evidence of crystalline drug.

In examples 1 and 3 (example 3 used 5% w/v mannitol), a secondary drying temperature of 40° C. under a pressure of 100 mTorr for up to 24 hours was used to reduce residual t-butyl alcohol to levels of less than 1% of the final weight of solids. Examples 4-8 illustrate other pre-lyophilization formulations of the invention.

### Example 4

| | |
|---|---|
| CCI-779 | 100 mg |
| dehydrated alcohol | q.s. 1 mL |

As used in these examples, dehydrated alcohol, USP, consists of not less than 98% by volume of ethanol (ethyl alcohol).

### Example 5

| | |
|---|---|
| CCI-779 | 25 mg |
| BHT | 0.2 mg |
| Citric acid anhydrous | 0.05 mg |
| dehydrated alcohol | q.s. 1.0 mL |

### Example 6

| | |
|---|---|
| CCI-779 | 25 mg |
| d,l-alpha tocopherol | 0.75 mg |
| glycine HCl | 0.1 mg |
| mannitol | 20 mg |
| 0.01 N. HCl | adjust pH to 5.5 q.s. |
| 60% t-butyl alcohol in water | q.s. 1.0 mL |

### Example 7

| | |
|---|---|
| CCI-779 | 25 mg |
| BHT | 0.75 mg |
| glycine HCl | 0.1 mg |
| mannitol | 20 mg |
| 0.01 N. HCl | adjust pH to 5.5 q.s. |
| 60% t-butyl alcohol in water | q.s. 1.0 mL |

### Example 8

| | |
|---|---|
| CCI-779 | 25 mg |
| d,l-alpha tocopherol | 0.75 mg |
| Citric acid anhydrous | 0.1 mg |
| mannitol | 20 mg |
| 0.01 N. HCl | adjust pH to 5.5 |
| | q.s. |
| 60% t-butyl alcohol in | q.s. 1.0 mL |
| water | |

The above solutions will be filtered, filled into glass vials and freeze dried to remove the alcohol-water mixture. The vials will be back-filled with nitrogen gas prior to stoppering. X-ray diffraction patterns are anticipated to indicate that the resulting materials are largely amorphous. The freeze-dried materials are expected to retain potency after several months storage at 40° C.

Examples 9 and 10 illustrate reconstitution of the lyophilized CCI-779 formulae for administration by parenteral routes.

### Example 9

| | |
|---|---|
| Polysorbate 80 | 8% w/v |
| Dehydrated alcohol | 39.5% w/v |
| Water for injection | q.s. 100% |

The above diluent was added to Example 3 to produce a 10 mg/mL reconstituted solution of CCI-779. The reconstituted solution could be diluted 1:10 with 0.9% sodium chloride injection to produce a mixture that was free of visual precipitates.

In some instances, a diluent with a higher water content could be used to reconstitute freeze dried material at a concentration that is lower than the pre-lyophilized solution:

### Example 10

| | |
|---|---|
| Polysorbate 80 | 5% w/v |
| Water for injection | q.s. |

The above diluent was added to Example 2 to produce a 5 mg/mL reconstituted solution of CCI-779. The solution could potentially be injected directly or diluted into 0.9% sodium chloride injection for intravenous infusion.

The documents identified herein are incorporated by reference. A variety of modifications to the techniques and components described herein will be readily apparent to one of skill in the art and are encompassed within the scope of the following claims.

1. A solution useful for preparing freeze-dried CCI-779, said solution comprising 0.1 mg/mL to 250 mg/mL CCI-779 and at least 30% v/v solvent selected from the group consisting of dimethylsulfoxide, acetonitrile, ethanol, isopropanol, t-butyl alcohol, and blends thereof that optionally further contain water.

2. A solution according to claim 1, wherein said solution comprises 40% v/v water and has a pH in a range of 4 to 6.

3. A solution according to claim 1, wherein said solution comprises 40% to 70% v/v solvent.

4. A solution according to claim 1, wherein said solvent comprises t-butyl alcohol.

5. A solution according to claim 1, wherein the solvent is ethanol.

6. A solution according to claim 4, wherein said solvent comprises t-butyl alcohol is in the range of 40 to 60 v/v % t-butyl alcohol in water.

7. A solution according to claim 1 containing water wherein the pH is about 5.5.

8. A solution according to claim 1 comprising 10 mg/mL to 100 mg/mL CCI-779.

9. A solution according to claim 1, wherein said solution further comprises 2 to 5% w/v of a bulking agent.

10. A solution according to claim 9, wherein said bulking agent is mannitol.

11. A solution according to claim 1, wherein said solution further comprises an antioxidant.

12. A method of preparing a lyophilized CCI-779 formulation, said method comprising the step of freeze-drying a solution according to claim 1.

13. A method of preparing a lyophilized CCI-779 formulation, said method comprising the steps of:
(a) preparing a solution having a pH in the range of 4 to 6 and comprising 10 mg/mL to 100 mg/mL CCI-779, 2 to 5% w/v mannitol, and t-butyl alcohol in water; and
(b) freeze-drying said solution to form lyophilized CCI-779.

14. A lyophilized CCI-779 formulation formed by freeze-drying a solution according to claim 1.

15. A method of preparing CCI-779 for delivery in liquid form, said method comprising the step of reconstituting CCI-779 with parenterally acceptable solvent to form a concentrate CCI-779 solution and mixing said concentrate solution with a diluent comprising water to form a liquid dosage form of CCI-779.

16. A method according to claim 15, wherein said diluent further comprises 5 to 8% w/v polysorbate 80.

17. A method solution according to claim 15, wherein said diluent further comprises dehydrated alcohol.

18. A method according to claim 15, wherein said concentrate solution is diluted 1:9 with 0.9% sodium chloride solution.

19. A liquid dosage form of CCI-779 formed according to the method of claim 15.

20. A method of enhancing storage stability of CCI-779, said method comprising the step of lyophilizing a solution comprising 25 mg/mL to 100 mg/mL CCI-779, t-butyl alcohol, and a pH of 4 to 6.

21. A method according to claim 20, wherein said solution further comprises 2 to 5% w/v mannitol.

22. A kit comprising a container for the lyophilized CCI-779 according to claim 14 and a solvent for reconstitution thereof.
